(19) United States
(12) Patent Application Publication
(10) Pub. No.: US 2007/0032813 A1
(43) Pub. Date: Feb. 8, 2007

(54) Lancing device with pivoting end cap

(75) Inventors: Stephen J. Flynn, Peachtree City, GA (US); Richard W. LeVaughn, Newnan, GA (US); Chirstopher J. Ruf, Marietta, GA (US); Gwenn E. Kennedy, Ellenwood, GA (US); Michael V. Lipoma, Villa Rica, GA (US); Juan D. Salleras, Alpharetta, GA (US)

Correspondence Address: GARDNER GROFF SANTOS & GREENWALD, P.C., 2018 POWERS FERRY ROAD, SUITE 800, ATLANTA, GA, 30339, US

(73) Assignee: Facet Technologies, LLC, Marietta, GA (US)

(21) Appl. No.: 10/572,270

(22) PCT Filed: Jul. 28, 2004
(86) PCT No.: PCT/US04/24394

§ 371 Date: Mar. 15, 2006

Related U.S. Application Data
(60) Provisional application No. 60/504,009, filed on Sep. 18, 2003.

(51) Int. Cl. A61B 17/32

(52) U.S. Cl. 606/181

(57) ABSTRACT

An endcap for a lancing device, having a base portion and pivoting tip member pivotally mounted to the base portion. The base portion and the pivoting tip member each having openings therethrough which, when aligned, allow passage of a lancet tip. After lancing the skin at a sampling site, the lancing device is rocked back and forth, with a contact face of the pivoting tip member maintained in contact with the skin surrounding the sampling site to enhance sample collection and prevent premature wound closure.

### CROSS-REFERENCE TO RELATED APPLICATION

This application claims the benefit of U.S. Provisional Patent Application Ser. No. 60/504,009, filed Sep. 18, 2003, the entirety of which is hereby incorporated herein by reference for all purposes.

### TECHNICAL FIELD

The present invention relates generally to medical devices and procedures, and more particularly to lancing devices for sampling blood or other bodily fluids. The invention more particularly relates to an end cap for use in conjunction with a lancing device that allows for the collection of an increased volume of blood or bodily fluid to be sampled.

### BACKGROUND OF THE INVENTION

Many medical procedures require puncturing of the skin, and sometimes underlying tissues, of a human or animal subject. For example, a sharp lancet tip is commonly used to puncture the subject's skin for sampling of blood or other body fluid, as for example in blood glucose monitoring by diabetics. Generally, lancet blades or needles are much thinner than traditional hypodermic syringe needles, and therefore result in less pain to the patient. A lancet having a sharp lancing tip typically is mounted, permanently or releasably, to the drive mechanism of a lancing device. The drive mechanism includes one or more springs, cams, motors, or other mechanism(s) for moving the lancing tip from a retracted position shielded within a housing of the lancing device to an extended position outside of the housing for lancing the subject's skin. The lancing device may also include a cocking mechanism for arming the drive mechanism, and/or a triggering mechanism for firing the device. The housing of the lancing device typically includes an endcap with an opening through which the lancing tip extends in its extended position. The endcap may be releasably or permanently attached to the remainder of the housing, or can comprise an integral part of the housing.

One common problem that has been found to exist with many known lancets and lancing devices is the closure of the puncture site before an adequate volume of blood has been collected. Premature closure of the puncture requires additional blood to be drawn from the patient, often in a separate and sometimes less desirable location, resulting in an increase in pain and stress to the subject. Costs also increase, as multiple lancets are required to acquire an adequate sample. This is particularly the case when alternate sampling sites other than the fingertips, such as for example the forearm or earlobe, are lanced. Lancing at such alternate sampling sites may be desirable because repeated lancing of the fingertips can result in callous formation, leading to less consistent sampling and decreased sensitivity of the fingertips. In addition, the fingertips have a greater density of nerve endings than alternate sampling sites, resulting in greater pain sensation from fingertip lancing than lancing at alternate sites. To increase patient compliance with a prescribed sampling regimen, it is desirable to minimize pain resulting from the lancing operation. However, because alternate sites typically contain a lower density of capillaries or a deeper capillary bed than the fingertips, alternate sampling sites often yield insufficient sample sizes or are prone to premature closure.

Attempts have been made to provide lancing devices that inhibit premature closure of the puncture site and yield increased sample size. Such attempts have included the use of a stimulator member slideably mounted to the housing for cyclically pumping around the puncture site to “milk” the wound. Although utilizing the aforementioned pumping sequence may allow a larger sample to be collected before closure of the puncture site, occasionally a sample is still insufficient, and another lancing operation to collect blood or analytic fluid is required. Additionally, the repeated application and release of pressure from such pumping can result in contact between the sample and the lancing device, smearing and/or contaminating the sample and necessitating another lancing operation, and/or can lead to bruising of the tissue surrounding the sampling site. Also, the provision of a translationally mounted stimulator member results in increased complexity and cost of the lancing device.

Thus it can be seen that needs exist for improved lancing devices and methods to facilitate increased sample volume of blood or other analytic fluid, and to prevent premature closure of the sampling site. Needs further exist for such mechanisms and methods that are readily adaptable to current lancing devices and procedures, and which can be utilized for lancing at fingertip as well as alternate sample collection sites.

### SUMMARY OF THE INVENTION

In example forms, the present invention is an endcap for a lancing device that enables collection of an increased volume of blood or other body fluid to be sampled from a sampling site of a human or animal subject. The device of the present invention is suitable for use at fingertip sampling sites, but is also well suited for use at alternate sampling sites such as the forearm and/or earlobe. In further embodiments, the invention is a sampling method for increasing collected sample size of blood or other body fluid from a sampling site.

In one aspect, the present invention is an endcap for a lancing device. The endcap preferably includes a base member having a first end for connection to the lancing device, and a pivoting tip member with a contact face having an opening therethrough, wherein the pivoting tip member is pivotally coupled to the base member.

In another aspect, the invention is a lancing device for collecting a sample of body fluid from a sampling site on the skin of a subject. The lancing device preferably includes a lancet and a housing comprising an endcap, the lancet being movable between a first position within the housing and a second position wherein at least a sharp tip portion of the lancet extends out of the housing. The endcap preferably includes a base portion and a pivoting tip member pivotally coupled to the base member.

In still another aspect, the invention is a method of lancing skin to collect a fluid sample, the method including the steps of placing a contact face of a pivoting tip member portion of a lancing device against a subject's skin at a sampling site; lancing the skin at the sampling site; and rocking the lancing device to cause the lancing device to pivot relative to the pivoting tip member, with the contact face maintained in contact with the skin around the sampling site.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING

FIG. 1 shows a perspective view of an endcap for a lancing device according to an example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

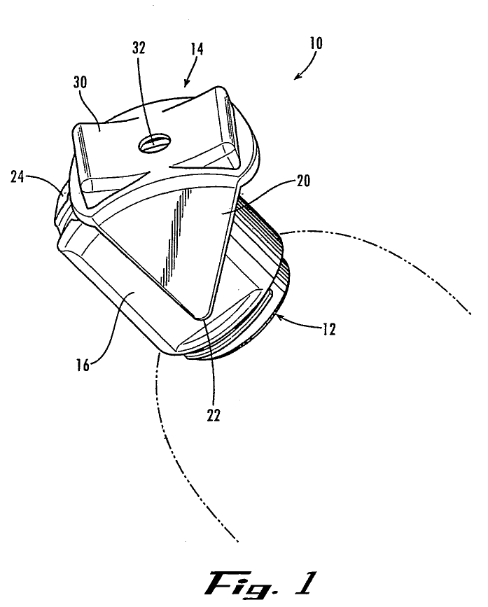

With reference now to the drawing, FIG. 1 shows an example embodiment of an endcap 10 for a lancing device according to the present invention. The endcap 10 preferably comprises a first end 12 for releasable or permanent attachment to the housing of a lancing device. Alternatively, the endcap forms an integral part of the housing of the lancing device. The endcap 10 can be adapted for use with any of a variety of standard, commercially-available or later-developed lancing devices, by appropriate configuration of coupling or attachment features of the first end 12. The endcap is preferably formed of an engineering or medical-grade polymer, plastic, or other material.

The endcap 10 preferably comprises a base member 16 that is attached at its proximal end to the lancing device housing, and a pivoting tip member 20 that is pivotally connected at its proximal end to the base member by a pivotal joint 22, such as one or more hinge pins, pin and socket couplings, or the like. The distal end of the base member 16 preferably comprises an arcuate or curved outer face 24 along which the inner face of the pivoting tip member 20 moves as it pivots. The arcuate outer face 24 preferably defines a slot or opening for permitting passage of the tip of a lancet therethrough. The distal end of the pivoting tip member 20 preferably defines a contact face 30 having an opening 32 therethrough for permitting passage of the tip of a lancet when the opening 32 is aligned with the slot or opening in the outer face 24 of the base member 16. The contact face 30 is preferably concave for more securely receiving a section of skin surrounding the sampling site, but alternatively may be flat or convex.

In a preferred method of operation according to the present invention, the opening 32 is aligned with the slot or opening in the outer face 24 of the base member 16, the contact face 30 is pressed against the skin around the sampling site, and the device is triggered to lance the skin, penetrating the skin surface to form a wound at the sample site. The lancing device is then rocked back and forth with the contact face 30 maintained against the skin around the sampling site. As the device is rocked back and forth, the pivoting tip member 20 pivots relative to the remainder of the lancing device, thereby helping maintain a fixed position of contact between the contact face 30 and the skin to prevent smearing or contamination of the sample that might result if the lancing device slipped along the skin surface, but alternating the point at which pressure is applied around the sampling site. This alternating application of pressure to different locations on the skin along either side of the sampling site directs blood toward the wound and helps prevent premature closure of the wound, thereby enhancing the sample size.

While the invention has been described with reference to preferred and example embodiments, it will be understood by those skilled in the art that a variety of modifications, additions and deletions are within the scope of the invention, as defined by the following claims.

What is claimed is:

1. An endcap for a lancing device, the endcap comprising a base member having a first end for connection to the lancing device, and a pivoting tip member comprising a contact face having an opening therethrough, wherein the pivoting tip member is pivotally coupled to the base member.

2. The endcap of claim 1, wherein the contact face is concave.

3. The endcap of claim 1, wherein the pivoting tip member is connected to the base member by two pin connections on opposite sides of the endcap.

4. The endcap of claim 1, wherein the base member comprises an arcuate distal end.

5. The endcap of claim 1, wherein the arcuate distal end of the base member comprises a slotted opening for alignment with the opening in the contact face of the pivoting tip member to allow passage of the sharp tip of a lancet.

6. A lancing device for collecting a sample of body fluid from a sampling site on the skin of a subject, said lancing device comprising a lancet and a housing comprising an endcap, the lancet being movable between a first position within the housing and a second position wherein at least a sharp tip portion of the lancet extends out of the housing, and wherein the endcap comprises a base portion and a pivoting tip member pivotally coupled to the base member.

7. The lancing device of claim 6, wherein the pivoting tip member comprises a contact face having an opening therethrough.

8. The lancing device of claim 7, wherein the base member comprises an arcuate distal end.

9. The lancing device of claim 8, wherein the arcuate distal end of the base member comprises a slotted opening for alignment with the opening in the contact face of the pivoting tip member to allow passage of the sharp tip of a lancet.

10. The lancing device of claim 7, wherein the contact face is concave.

11. The lancing device of claim 6, wherein the pivoting tip member is connected to the base member by two pin connections on opposite sides of the endcap.

12. A method of lancing skin to collect a fluid sample, the method comprising:
placing a contact face of a pivoting tip member portion of a lancing device against a subject's skin at a sampling site;
lancing the skin at the sampling site; and
rocking the lancing device to cause the lancing device to pivot relative to the pivoting tip member, with the contact face maintained in contact with the skin around the sampling site.
